(19) United States
(12) Patent Application Publication
(10) Pub. No.: US 2004/0204475 A1
(43) Pub. Date: Oct. 14, 2004

(54) Pharmaceutical combination

(76) Inventor: Michael John Humphrey, Sandwich (GB)

Correspondence Address: WARNER-LAMBERT COMPANY, 2800 PLYMOUTH RD, ANN ARBOR, MI, 48105, US

(21) Appl. No.: 10/819,604

(22) Filed: Apr. 7, 2004

Related U.S. Application Data
(60) Provisional application No. 60/469,710, filed on May 12, 2003.
(60) Provisional application No. 60/489,943, filed on Jul. 22, 2003.

(30) Foreign Application Priority Data
Apr. 11, 2003 (GB) 0308469.6
May 30, 2003 (GB) 0312479.9

(51) Int. Cl. A61K 31/405

(52) U.S. Cl. 514/414

(57) ABSTRACT

The present invention provides a combination of (a) eletriptan, or a pharmaceutically acceptable salt thereof, and (b) sodium bicarbonate. The combination provides a rapid absorption of eletriptan when taken orally. The combination is useful for the treatment or prevention of a disease for which a 5-HT₁ agonist is indicated, particularly for the treatment of migraine or for the prevention of migraine recurrence.

This application is a United States utility application, which claims the benefit of priority to United Kingdom application Serial No. 0308469.6 filed Apr. 11, 2003, and 0312479.9 filed May 30, 2003, and U.S. provisional application Ser. No. 60/489,943 filed Jul. 22, 2003.

The present invention relates to a combination comprising eletriptan, or a pharmaceutically acceptable salt thereof, and sodium bicarbonate, the use of such a combination for the treatment or prevention of a disease for which a 5-HT₁ agonist is indicated and formulations and products comprising such a combination.

Eletriptan, 3-{[1-methylpyrrolidin-2(R)-yl]methyl}-5-(2-phenylsulfonylethyl)-1H-indole, and a process for its manufacture, are disclosed in U.S. Pat. No. 5,607,951.

Eletriptan is a potent 5-HT₁ agonist and may be used in the treatment of hypertension, depression, anxiety, eating disorders, emesis, obesity, drug abuse, cluster headache, migraine (including menstrual migraine and recurrent migraine), pain, chronic paroxysmal hemicrania or headache associated with vascular disorders (see U.S. Pat. No. 5,607,951, WO-A-96/06842 and WO-A-00/32589).

Eletriptan is useful in the treatment of migraine in children, in the treatment of mild migraine, menstrual migraine and early migraine and in the treatment of post-traumatic head and neck injury, mixed headaches and tension headaches.

As disclosed in WO-A-00/06161, eletriptan is also useful in the prevention of migraine recurrence.

Pharmaceutical compositions suitable for the oral administration of eletriptan, or a pharmaceutically acceptable salt thereof, for example tablets or capsules, prepared by conventional means with pharmaceutically acceptable excipients such as binding agents, fillers, lubricants, disintegrants or wetting agents, are known.

Such compositions dissolve in the gastrointestinal tract and the released drug is absorbed into the blood stream after a certain time lag. For instance, when eletriptan is administered in the form of the α-polymorphic hydrobromide salt disclosed in WO-A-96/06842, the time period that elapses between administering a standard tablet and observing maximal plasma concentrations of the drug in the bloodstream (Tₘₐₓ) ranges from about 1 hour in fasted subjects to up to about 4 hours in fed subjects.

Problematically, a migraine attack can further increase Tₘₐₓ, delaying drug absorption, by causing gastric stasis. For instance, in one study in which 30 mg eletriptan was administered to 34 fasted subjects, with and without migraine, Tₘₐₓ was significantly slowed in patients with migraine (Tₘₐₓ2.8±2.1 h) when compared with migraine-free patients (Tₘₐₓ1.3±1.3 h).

Clearly, there exists a need to minimise the time lag between the administration of eletriptan, or a pharmaceutically acceptable salt thereof, orally and the relief of symptoms brought about by the delivery of the drug via the bloodstream to important tissues.

It has now been surprisingly found that when eletriptan, or a pharmaceutically acceptable salt thereof, is administered in combination with sodium bicarbonate, via the oral route, the time lag between oral administration and maximal plasma levels of drug is dramatically reduced, in some cases to as little as 30 minutes. The advantage to the patient arising from the correspondingly swift amelioration of symptoms is self-evident.

The present invention therefore provides a combination of eletriptan, or a pharmaceutically acceptable salt thereof, and sodium bicarbonate.

The invention further provides a combination of eletriptan, or a pharmaceutically acceptable salt thereof and sodium bicarbonate, for use as a medicament.

The invention further provides a combination of eletriptan, or a pharmaceutically acceptable salt thereof and sodium bicarbonate, for use in the treatment or prevention of a disease for which a 5-HT₁ agonist is indicated, particularly for use in the treatment of migraine or in the prevention of migraine recurrence.

The invention further provides the use of (a) eletriptan, or a pharmaceutically acceptable salt thereof, or (b) sodium bicarbonate for the manufacture of a medicament for the treatment or prevention of a disease for which a 5-HT₁ agonist is indicated, particularly for the treatment of migraine or for the prevention of migraine recurrence, wherein (a) and (b) are to be taken separately, sequentially or simultaneously by the oral route.

The invention further provides a method of treating or preventing a disease for which a 5-HT₁ agonist is indicated, particularly a method of treating migraine or preventing migraine recurrence, in a mammal, including a human being, including simultaneous, separate or sequential administration by the oral route of (a) an effective amount of eletriptan, or a pharmaceutically acceptable salt thereof and (b) an effective amount of sodium bicarbonate.

The invention further provides a pharmaceutical composition, suitable for oral administration, including eletriptan, or a pharmaceutically acceptable salt thereof, sodium bicarbonate and a pharmaceutically acceptable excipient, diluent or carrier.

The invention further provides a product containing (a) eletriptan, or a pharmaceutically acceptable salt thereof and (b) sodium bicarbonate as a combined preparation for simultaneous, separate or sequential use, via oral administration, in the treatment or prevention of a disease for which a 5-HT₁ agonist is indicated, particularly in the treatment of migraine or in the prevention of migraine recurrence.

Pharmaceutically acceptable salts of eletriptan include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

Preferred salts of eletriptan in the context of the present invention are the hydrobromide and hemisulphate salts (see WO-A-96/06842 and WO-A-01/23377), especially the hydrobromide salt.

A pharmaceutically acceptable salt of eletriptan may be readily prepared by mixing together eletriptan and the desired acid or base, as appropriate. The salt may precipitate from solution and be collected by filtration or may be recovered by evaporation of the solvent.

Pharmaceutically acceptable solvates of eletriptan, including hydrates and solvates wherein the solvent of crystallization may be isotopically substituted (e.g. D₂O, d₆-acetone, d₆-DMSO), are also within the scope of the invention.

Also within the scope of the invention are clathrates, drug-host inclusion complexes wherein, in contrast to the aforementioned solvates, the drug and host are present in stoichiometric or non-stoichiometric amounts. For a review of such complexes, see J. Pharm. Sci., 64 (8), 1269-1288 by Haleblian (August 1975).

Any polymorph of eletriptan, or a pharmaceutically acceptable salt thereof, may be used in the invention. Particularly preferred is the α-polymorphic form of eletriptan hydrobromide described in WO-A-96/06842 and the polymorphic form of eletriptan hemisulphate defined by the claims of WO-A-01/23377, particularly the former.

Also within the scope of the invention are so-called “prodrugs” of eletriptan and its pharmaceutically acceptable salts. Thus certain derivatives of eletriptan and its salts, which have little or no pharmacological activity themselves can, upon administration into or onto the body, be converted, for example by metabolism or hydrolytic cleavage, to eletriptan itself. Such derivatives are referred to as “prodrugs”. Further information on the use of prodrugs may be found in ‘Prodrugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in eletriptan with certain moieties known to those skilled in the art as “pro-moieties” as described, for example, in “Design of Prodrugs” by H Bundgaard (Elsevier, 1985).

In the case of eletriptan, prodrugs of particular importance are derivatives of the indole NH group, for example by replacement of the hydrogen atom with (C₁-C₁₀ )alkanoyl.

The present invention also includes all pharmaceutically acceptable isotopic variations of eletriptan and its pharmaceutically acceptable salts. An isotopic variation is defined as one in which at least one atom is replaced by an atom having the same atomic number, but an atomic mass different from the atomic mass usually found in nature. Examples of possible isotopes include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution with isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variants of eletriptan and its salts can generally be prepared by conventional techniques known to those skilled in the art or by processes analogous to those described in the art for the preparation of eletriptan using appropriate isotopic variants of suitable reagents.

Eletriptan, or a pharmaceutically acceptable salt thereof, and sodium bicarbonate (henceforth, ‘compounds of the invention’) may be freeze-dried, spray-dried, or evaporatively dried to provide a solid plug, powder, or film of crystalline or amorphous material. Microwave or radio frequency drying may be used for this purpose.

The compounds of the invention may be administered alone but will generally be administered as a formulation in association with one or more pharmaceutically acceptable excipients. The term “excipient” is used herein to describe any ingredient other than the compound of the invention.

The compounds of the invention are administered orally. Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

Tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form. It is preferred that tablets made according to the present invention should comprise microcrystalline cellulose as a disintegrant.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. It is preferred that tablets made according to the present invention should comprise microcrystalline cellulose as a binder.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet. It is preferred that tablets made according to the present invention should comprise magnesium stearate as a lubricant.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, New York, N.Y., 1980 (ISBN 0-8247-6918-X).

The compounds of the invention may be combined with soluble macromolecular entities such as cyclodextrin or polyethylene glycol-containing polymers to improve their solubility, dissolution rate, taste-masking, bioavailability and/or stability. Both inclusion and non-inclusion complexes may be used. As an alternative to direct complexation with the drug, a cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO-A-91/11172, WO-A-94/02518 and WO-A-98/55148.

For administration to human patients, the total daily dose of eletriptan is typically in the range from 0.1 mg to 4 mg/kg, in single or divided doses. A single first dose of 40 mg is currently recommended. The physician, in any event, will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Thus, tablets or capsules according to the invention will typically contain from 5 to 240 mg, preferably from 5 to 100 mg of eletriptan, for administration singly or two or more at a time, as appropriate. In especially preferred embodiments of the present invention, tablets comprising (a) 40 mg of eletriptan in the form of its hydrobromide salt for administration singly or (b) 20 mg of eletriptan in the form of its hydrobromide salt for administration two at a time are provided.

Enough sodium bicarbonate should preferably be administered to obtain a duodenal concentration approximately isotonic with serum (150 mM). So, for instance, if the combination of the invention is to be taken with half a tumbler of water (100 ml) in fasted volunteers, 1260 mg of sodium bicarbonate would be required to provide such an isotonic solution. Such a dose of sodium bicarbonate is too large to be incorporated conveniently into a single tablet. It is thus preferred that, where the combination is to be administered simultaneously in the form of a tablet, tablets comprising about 630 mg sodium bicarbonate for administration two at a time are provided. Alternatively, if the combination of the invention is to be taken with 50 ml of water, a single tablet containing about 630 mg of sodium bicarbonate is preferred.

In one embodiment, a combination or formulation according to the invention (especially a tablet) comprises greater than 50% (preferably greater than 55%) by weight of sodium bicarbonate.

It is preferred that the eletriptan, or salt thereof, and bicarbonate are administered simultaneously, preferably in the form of a tablet containing both compounds.

Preferably, such a tablet contains from 20 to 40 mg of eletriptan (in the form of its hydrobromide salt) in combination with from 300 to 1300 mg, most preferably about 630 mg, of sodium bicarbonate.

Preferably, such a tablet contains from 20 to 40 mg, most preferably 20 or 40 mg, of eletriptan (in the form of its hydrobromide salt) in combination with about 630 mg of sodium bicarbonate.

The present combination of eletriptan, or a pharmaceutically acceptable salt thereof, and sodium bicarbonate may also be administered in combination with one or more further active substances. Examples of such further active substances include:

(a) a further antimigraine drug, particularly a further triptan such as sumatriptan, naratriptan, rizatriptan, almotriptan, avitriptan, frovatriptan or zolmitriptan;

(b) a prokinetic agent such as those mentioned in WO-A-02/070070, e.g. metaclopramide;

(c) an alkylxanthine compound such as caffeine, theophylline or theobromine, particularly caffeine; and

(d) a non-steroidal anti-inflammatory drug (NSAID) such as a COX-2 inhibitor, e.g. celecoxib or valdecoxib.

In a further embodiment of the invention, the combination of eletriptan, or a salt thereof, and sodium bicarbonate, may also further comprise a pharmaceutically acceptable acidic agent such that on exposure to an aqueous medium the combination will dissolve in an effervescent manner. Examples of suitable pharmaceutically acceptable acidic agents include adipic acid, ascorbic acid, citric acid, fumaric acid, glycolic acid, lactic acid, maleic acid, alic acid, succinic acid and tartaric acid. Citric acid and adipic acid are preferred.

It is to be appreciated that all references herein to treatment include curative, palliative and prophylactic treatment.

The combination of the present invention may be conveniently presented in tablet form along with a suitable quantity of water with which the tablet or tablets can be taken.

The invention therefore further provides a product comprising (a) eletriptan, or a pharmaceutically acceptable salt thereof, (b) sodium bicarbonate, (c) water and (d) suitable packaging.

Preferably, such a product contains either (a) two tablets, each containing 20 mg of eletriptan (in the form of its hydrobromide salt) and about 630 mg of sodium bicarbonate, and about 100 ml of water or (b) one tablet containing 40 mg of eletriptan (in the form of its hydrobromide salt) and about 630 mg of sodium bicarbonate, and about 50 ml of water.

Whilst the invention has been described above with reference solely to eletriptan, or a pharmaceutically acceptable salt thereof, it has been shown that any 5-HT₁ agonist can in principle be combined with sodium bicarbonate in order to obtain an increased rate of gastrointestinal absorption when delivered by the oral route. Such 5-HT₁ agonists include almotriptan, alnatriptan, avitriptan, frovatriptan, naratriptan, rizatriptan, sumatriptan, zolmitriptan.

The invention therefore further provides a non-effervescent composition adapted for oral administration and gastrointestinal drug absorption comprising a 5-HT₁ agonist and sodium bicarbonate.

In this context, non-effervescent means that when the composition is dissolved in water at pH 7, no effervescence is observed.

All preferred features relating to a combination of eletriptan and sodium bicarbonate, as described above, apply equally to the non-effervescent composition comprising a 5-HT₁ agonist and sodium bicarbonate. In particular, in one embodiment the composition comprises greater than 50% (preferably greater than 55%) by weight of sodium bicarbonate.

The following Examples illustrate the invention.

### EXAMPLES 1-4

### Tablets Comprising Eletriptan Hydrobromide and Sodium Bicarbonate

A mixture of eletriptan hydrobromide (24.24 mg, equivalent to 20 mg of eletriptan free base), sodium bicarbonate (USP, 630 mg) and microcrystalline cellulose (Avicel PH 102, Ph. Eur., 137.76 mg) were blended for 10 minutes, passed through a 500 μM screen and re-blended for a further 10 minutes. Screened intragranular magnesium stearate (Ph. Eur., 8 mg) was added and the mixture was lubricated for 5 minutes. The blend so obtained was found to have a particle size range from approximately 200 μm to 1 mm and to have excellent flow properties. The blend was made into a tablet by direct compression. The tablets so produced had excellent weight uniformity and friability.

Surprisingly, it was found that eletriptan, at a tablet composition of 2.5% w/w, was able to confer extra strength to the tablets, as demonstrated by an increase of 0.7 kp in tablet hardness relative to tablets made from a placebo blend.

Examples 2 to 4 in Table 1 describe further tablets made according to the method of Example 1. In the case of Examples 3 and 4, the lactose and croscarmellose sodium were added to the initial mixture before blending.

TABLE 1

| Ingredient | Example 2 | | Example 3 | | Example 4 | |
|---|---|---|---|---|---|---|
| Eletriptan hydrobromide | 48.48 | mg | 24.24 | mg | 48.48 | mg |
| Microcrystalline cellulose | 113.52 | mg | 248.07 | mg | 229.89 | mg |
| Sodium bicarbonate | 630 | mg | 630 | mg | 630 | mg |
| Lactose NF | — | | 82.69 | mg | 76.63 | mg |
| Croscarmellose sodium | — | | 5 | mg | 5 | mg |
| Magnesium stearate | 8 | mg | 10 | mg | 10 | mg |

### EXAMPLE 5

### Pharmacokinetic Evaluation of an Eletriptan Hydrobromide/Sodium Bicarbonate Tablet Relative to a Standard Eletriptan Hydrobromide Tablet

The objective of this study was to measure plasma concentrations of eletriptan in four beagle dogs following administration of tablets comprising eletriptan with and without sodium bicarbonate as single oral doses and thus to determine the effect of sodium bicarbonate on the rate of absorption and exposure of eletriptan.

The following two formulations were compared:

(a) a commercially available 20 mg tablet comprising eletriptan hydrobromide taken with 50 ml water; and

(b) the tablet of Example 1 taken with 50 ml water.

Four beagles were used in the study, two male and two female. Each beagle received one of study formulations and blood samples were then taken during a subsequent 24 hour period (every 5 minutes for the first 45 minutes) to monitor plasma levels of eletriptan. The beagles subsequently received the alternative formulation and blood samples were taken in an identical fashion. At least one week was allowed to elapse between treatments.

The results of the study are listed in Table 2. Mean values for the key pharmacokinetic parameters Tₘₐₓ (time to maximum plasma concentration), Cₘₐₓ (highest plasma concentration) and AUC (total exposure to drug—area under the curve from zero to infinity) are given as an average of the values for each dog. In the case of formulation (b), one dog vomited 15 minutes after treatment, leading to anomalous results which were not included in the calculations. Standard deviations from the mean values are also listed.

TABLE 2

| | |
|---|---|
| Formulation (a) | Formulation (b) |

| Pharmacokinetic | | Standard | | Standard |
|---|---|---|---|---|
| parameter | Mean | deviation | Mean | deviation |
| Tₘₐₓ (hours) | 2.8 | 2.2 | 1.2 | 0.3 |
| Cₘₐₓ (ng/ml) | 22.6 | 5.65 | 31.8 | 2.61 |
| AUC (ng · h/ml) | 207 | 57 | 179 | 3 |

The results show that administration of a tablet comprising 20 mg eletriptan and 630 mg sodium bicarbonate with 50 ml water results in a more rapid absorption of drug than administration of a commercial 20 mg eletriptan tablet with 50 ml water, as shown by a reduction in Tₘₐₓ from a mean of 2.8 hours (±2.2) to a mean of 1.2 hours (±0.3), i.e. an average reduction in Tₘₐₓ of 57%. There is a corresponding increase in Cₘₐₓ from a mean of 22.6 ng/ml to a mean of 31.8 ng/ml. Overall exposure, as measured by AUC is similar. Furthermore, inter-animal variability is greatly reduced following administration of formulation (b) comprising sodium bicarbonate as compared to administration of standard formulation (a).

1. A combination of (a) eletriptan, or a pharmaceutically acceptable salt thereof, and (b) sodium bicarbonate.

2. A combination as claimed in claim 1 wherein (a) is eletriptan hydrobromide or eletriptan hemisulphate.

3. A combination as claimed in claim 1 for use as a medicament.

4. A combination as claimed in claim 1 for use in the treatment or prevention of a disease for which a 5-HT₁ agonist is indicated, particularly for use in the treatment of migraine or in the prevention of migraine recurrence.

5. A method of treating or preventing a disease for which a 5-HT₁ agonist is indicated, particularly a method of treating migraine or preventing migraine recurrence, in a mammal, including a human being, including simultaneous, separate or sequential administration by the oral route of (a) an effective amount of eletriptan, or a pharmaceutically acceptable salt thereof and (b) an effective amount of sodium bicarbonate.

6. The method of claim 5 wherein (a) is eletriptan hydrobromide or eletriptan hemisulphate.

7. A pharmaceutical composition, adapted for oral administration, including a combination as defined in claim 1 and a pharmaceutically acceptable excipient.

8. A pharmaceutical composition, adapted for oral administration, including a combination as defined in claim 2 and a pharmaceutically acceptable excipient.

9. A pharmaceutical composition as claimed in claim 7 in the form of a tablet.

10. A pharmaceutical composition as claimed in claim 8 in the form of a tablet.

11. A pharmaceutical composition as claimed in claim 9 comprising eletriptan hydrobromide, sodium bicarbonate, microcrystalline cellulose and magnesium stearate.

12. A pharmaceutical composition as claimed in claim 10 comprising eletriptan hydrobromide, sodium bicarbonate, microcrystalline cellulose and magnesium stearate.

13. A package containing (a) eletriptan, or a pharmaceutically acceptable salt thereof and (b) sodium bicarbonate as a combined preparation for simultaneous, separate or sequential use, via oral administration, in the treatment or prevention of a disease for which a 5-HT₁ agonist is indicated, particularly in the treatment of migraine or in the prevention of migraine recurrence.

14. A package as claimed in claim 13 comprising the composition of claim 9 or 10.

15. A package as claimed in claim 13 which further comprises a quantity of water suitable for facilitating oral administration of the eletriptan, or salt thereof, and sodium bicarbonate.

16. A package as claimed in claim 15 comprising either (a) two tablets, each containing 20 mg of eletriptan (in the form of its hydrobromide salt) and about 630 mg of sodium bicarbonate, and about 100 ml of water or (b) one tablet containing 40 mg of eletriptan (in the form of its hydrobromide salt) and about 630 mg sodium bicarbonate and about 50 ml of water.

17. A non-effervescent composition adapted for oral administration and gastrointestinal drug absorption comprising a 5-HT₁ agonist and sodium bicarbonate.
